(19) United States
(12) Patent Application Publication
(10) Pub. No.: US 2014/0222065 A1
(43) Pub. Date: Aug. 7, 2014

(54) HEMOSTASIS DEVICES, COMPONENTS FOR HEMOSTASIS DEVICES, AND RELATED METHODS

(71) Applicant: ST. JUDE MEDICAL PUERTO RICO LLC, Caguas, PR (US)

(72) Inventor: Zachary J. Tegels, Minneapolis, MN (US)

(73) Assignee: St. Jude Medical Puerto Rico LLC, Caguas, PR (US)

(21) Appl. No.: 13/757,506

(22) Filed: Feb. 1, 2013

(51) Int. Cl. A61B 17/00

(52) U.S. Cl. CPC A61B 17/0057
USPC 606/213; 29/428

(57) ABSTRACT

A hemostasis device may comprise a collagen plug and a bioresorbable carrier. The collagen plug may comprise a body having a first end surface and an aperture extending through the body and the first end surface of the collagen plug. The bioresorbable carrier may comprise a tubular portion extending through the aperture of the body of the collagen plug, and a base portion extending radially from an end of the tubular portion, the base portion positioned adjacent to the first end surface of the collagen plug.

### TECHNICAL FIELD

The present disclosure relates generally to devices and methods for providing a hemostatic seal for tissue punctures, such as vascular punctures, and more particularly, to hemostasis devices, methods of manufacturing hemostasis devices, and components for hemostasis devices.

### BACKGROUND

Various surgical procedures are routinely carried out intravascularly or intraluminally. For example, in the treatment of vascular disease, such as arteriosclerosis, it is a common practice to access the artery and insert an instrument (e.g., a balloon or other type of catheter) to carry out a procedure within the artery. Such procedures usually involve the percutaneous puncture of the artery so that an insertion sheath may be placed in the artery and thereafter instruments (e.g., catheters) may pass through the sheath to an operative position within the artery. Intravascular and intraluminal procedures unavoidably present the problem of stopping the bleeding at the percutaneous puncture after the procedure has been completed and after the instruments (and any insertion sheaths used therewith) have been removed. Bleeding from puncture sites, particularly in the case of femoral arterial punctures, is typically stopped by utilizing vascular closure devices.

Collagen plugs are desirable for achieving hemostasis of a tissue puncture, as a collagen plug may remain within the patient and be absorbed by the patient's body over time. Additionally, collagen plugs may be provided with chemicals that facilitate blood coagulation. Complications may arise, however, with the use of collagen plugs.

One complication is when a collagen plug extends into the blood vessel, which may cause blood flow restriction, blood clotting within the vessel, collagen in the blood stream, and/or other complications. Additionally, it is difficult to retrieve a locating device through a collagen plug, as a locating device may not collapse properly against the relatively soft collagen.

### SUMMARY

One aspect of the present disclosure relates to a hemostasis device comprising a collagen plug and a bioresorbable carrier. The collagen plug may comprise a body having a first end surface and an aperture extending through the body and the first end surface of the collagen plug. The bioresorbable carrier may comprise a tubular portion and a base portion. The tubular portion of the bioresorbable carrier may extend through the aperture of the body of the collagen plug. The base portion of the bioresorbable carrier may extend radially from an end of the tubular portion.

An additional aspect, which may be combined with other aspects herein, relates to the base portion of the bioresorbable carrier being positioned adjacent to the first end surface of the collagen plug. The body of the collagen plug may further comprise a second end surface. The aperture extending through the body of the collagen plug may extend from the first end surface to the second end surface. The tubular portion of the bioresorbable carrier may extend through the aperture of the collagen plug from the first end surface to the second end surface. The tubular portion of the bioresorbable carrier may comprise a tapered central aperture, and the body of the collagen plug may be substantially cylindrical.

An additional aspect, which may be combined with other aspects herein, relates to the base portion of the bioresorbable carrier having a substantially circular outer circumference. The base portion of the bioresorbable carrier may be substantially flat, having substantially planar major surfaces. The base portion of the bioresorbable carrier may be concave, having dished major surfaces. Major surfaces of the base portion of the bioresorbable carrier may be oriented at a non-perpendicular angle relative to a longitudinal axis of the tubular portion. The bioresorbable carrier may comprise an integral valve within the tubular portion of the bioresorbable carrier for providing a hemostatic seal. The integral valve may be positioned at an end of the tubular portion proximate to the base portion of the bioresorbable carrier. The integral valve may comprise a plurality of flaps.

An additional aspect, which may be combined with other aspects herein, relates to the bioresorbable carrier being comprised of a bioresorbable polymer. An outer diameter of the tubular portion of the bioresorbable carrier may comprise surface features configured to maintain the position of the collagen plug relative to the bioresorbable carrier. The hemostasis device may further comprise a delivery tube, the bioresorbable carrier and collagen plug located in a distal end of the delivery tube. The base portion of the bioresorbable carrier may be elastically deformed within the distal end of the delivery tube.

One aspect of the present disclosure relates to a method of manufacturing a vascular closure device may comprise providing a collagen plug comprising a body having a first end surface and an aperture extending through the body and the first end surface of the collagen plug. The method may further comprise providing a bioresorbable carrier comprising a tubular portion and a base portion extending radially from an end of the tubular portion. The method may also comprise positioning the tubular portion of the bioresorbable carrier within the aperture of the collagen plug and positioning the base portion of the bioresorbable carrier adjacent to the first end surface of the collagen plug.

An additional aspect, which may be combined with other aspects herein, relates to the method further comprising forming an integral hemostatic seal within the tubular portion of the bioresorbable carrier. The method may comprise providing a bioresorbable carrier comprising a tubular portion with a tapered central aperture. The method may also comprise providing a bioresorbable carrier comprising a tubular portion and a concave base portion having dished major surfaces extending radially from an end of the tubular portion.

One aspect of the present disclosure relates to a component for a hemostasis device comprising a bioresorbable carrier. The bioresorbable carrier may comprise a tubular portion comprising an inner surface, an outer surface, a first end and a second end. The bioresorbable carrier may comprise a base portion extending radially from the second end of the tubular portion. The bioresorbable carrier may comprise a valve located within the tubular portion configured to facilitate the passage of an elongate device through the tubular portion and to provide a hemostatic seal of the tubular portion when an elongate device is not positioned through the valve. The base portion and the outer surface of the tubular portion may be configured to receive a collagen plug thereon.

One aspect of the present disclosure relates to a component for a hemostasis device comprising a bioresorbable carrier. The bioresorbable carrier may comprise a tubular portion, a base portion, and a valve. The tubular portion may comprise an inner surface, an outer surface, a first end and a second end. The base portion may extend radially from the second end of the tubular portion. The valve may be located within the tubular portion and configured to facilitate the passage of an elongate device through the tubular portion. The valve may also provide a hemostatic seal of the tubular portion when an elongate device is not positioned through the valve. Additionally, the base portion and the outer surface of the tubular portion may be configured to receive a collagen plug thereon.

### BRIEF DESCRIPTION OF THE DRAWINGS

The accompanying drawings illustrate various embodiments of the present methods and systems and are a part of the specification. The illustrated embodiments are merely examples of the present systems and methods and do not limit the scope thereof.

FIG. 1 is a perspective view of a hemostasis device according to an embodiment of the present disclosure.

FIG. 2 is a perspective view of a collagen plug of the hemostasis device of FIG. 1.

FIG. 3 is a top perspective view of a bioresorbable carrier of the hemostasis device of FIG. 1.

FIG. 4 is a bottom perspective view of the bioresorbable carrier of FIG. 3.

FIG. 5 is a cross-sectional view of the hemostasis device of FIG. 1.

FIG. 6 is a cross-sectional view of a hemostasis device including a bioresorbable carrier having a tubular portion with a textured outer surface, according to an additional embodiment of the present disclosure.

FIG. 7 is a cross-sectional view of a hemostasis device including a bioresorbable carrier having a dish-shaped base portion, according to an additional embodiment of the present disclosure.

FIG. 8 is a cross-sectional view of a hemostasis device including a bioresorbable carrier having a tapered tubular portion, according to an additional embodiment of the present disclosure.

FIG. 9 is a cross-sectional view of a hemostasis device including angled end surfaces, according to an additional embodiment of the present disclosure.

FIG. 10 is a partial cross-sectional side view of a hemostasis device including a collagen plug and bioresorbable carrier positioned within a delivery tube, according to an additional embodiment of the present disclosure, and the delivery tube positioned within a tissue tract of a patient.

FIG. 11 is a partial cross-sectional side view of the hemostasis device of FIG. 10 wherein the delivery tube has been retracted from the collagen plug and bioresorbable carrier.

FIG. 12 is a partial cross-sectional side view of the hemostasis device of FIG. 11 wherein a balloon locating device has been withdrawn from the hemostasis device.

FIG. 13 is a partial cross-sectional side view of the hemostasis device of FIG. 12 wherein a bioresorbable plug has been inserted into the tubular portion of the bioresorbable carrier.

Throughout the drawings, identical reference numbers designate similar, but not necessarily identical, elements.

### DETAILED DESCRIPTION

The devices and systems disclosed herein may be used to close or seal percutaneous punctures made through the body tissue of a patient to gain access to a body cavity of a patient. Access through these percutaneous punctures allows a physician to carry out various procedures in or through the body cavity for examination, surgery, treatment and the like. While not meant to be limiting, the systems are illustrated being used to seal percutaneous punctures that provide access to blood vessels in patients for various procedures. It will be appreciated that the systems are applicable to other procedures requiring sealing of a puncture through body tissue into a cavity including, for example, laparoscopic surgery and other surgery techniques using a relatively small incision.

The principles described herein may be used with any medical device. Therefore, while the description below is directed primarily to vascular procedures and certain embodiments of a vascular closure device, the methods and apparatus are only limited by the appended claims. Applications of closure devices including those implementing principles described herein include closure of a percutaneous puncture or incision in tissue separating two internal portions of a living body, such as punctures or incisions in blood vessels, ducts or lumens, gall bladders, livers, hearts, etc.

As used in this specification and the appended claims, the terms “engage” and “engagable” are used broadly to mean interlock, mesh, or contact between two structures or devices. Likewise “disengage” or “disengagable” means to remove or capable of being removed from interlock, mesh, or contact. A “tube” is an elongated device with a passageway. The passageway may be enclosed or open (e.g., a trough). A “lumen”, when referring to a bodily organ, refers to any open space or cavity in the bodily organ, especially in a blood vessel. The words “including” and “having,” as well as their derivatives, as used in the specification, including the claims, have the same meaning as the word “comprising.”

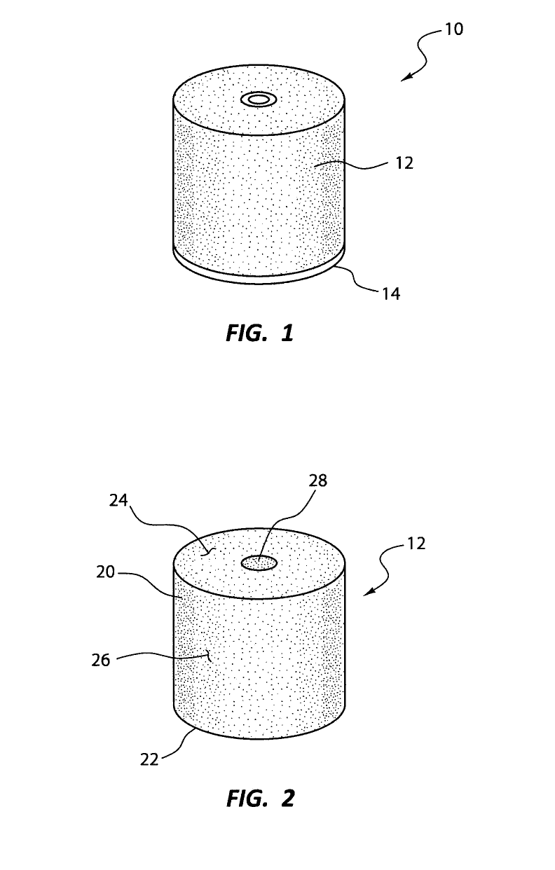

An embodiment of a hemostasis device 10 for closing a percutaneous puncture is shown in FIG. 1. The hemostasis device 10 comprises a collagen plug 12 positioned on a bioresorbable carrier 14.

As shown separately in FIG. 2, the collagen plug 12 may be generally cylindrical in shape. The body 20 of the collagen plug 12 may include a first end surface 22, a second end surface 24, and a side surface 26. In the embodiment shown in FIG. 2, the first end surface 22 and the second end surface 24 are substantially planar, and the side surface 26 is curved.

An aperture 28 may extend through the body 20 of the collagen plug 12, extending from the first end surface 22 to the second end surface 24. The aperture 28 may be centered in the body 20 of the collagen plug 12, extending along and coaxial with a longitudinal axis of the collagen plug 12. Optionally, the outer surfaces of the collagen plug 12 may be coated with human coagulation factors, such as fibrinogen and thrombin.

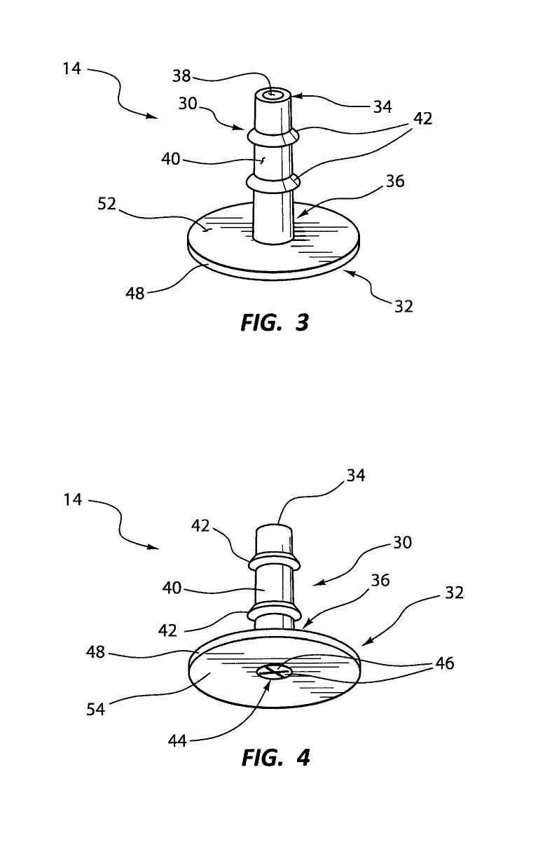

As shown separately in FIGS. 3 and 4, the bioresorbable carrier 14 may comprise a tubular portion 30 and a base portion 32. The tubular portion 30 may be configured generally as a round tube, the tubular portion 30 including a first end 34, a second end 36, an inner surface 38, and an outer surface 40. The outer surface 40 of the tubular portion 30 may include surface features 42, such as a ring with an angled surface (see FIGS. 3-5), a surface texture (see FIG. 6), one or more of a sharp, pointed or angled structure like a barb or barb-like ring, a faceted ring, or a ring or washer with top and bottom angled, contoured, or dished surfaces, any of which may facilitate the retention of the collagen plug 12 on the bioresorbable carrier 14.

In the embodiment shown in FIGS. 3 and 4, the base portion 32 may extend radially from the second end 36 of the tubular portion 30, having a generally annular, plate-like shape. The major surfaces 52, 54 of the base portion 32 may be substantially planar and may extend substantially perpendicular to a primary axis of the tubular portion 30. An outer circumference 48 of the base portion 32 may be generally circular in shape.

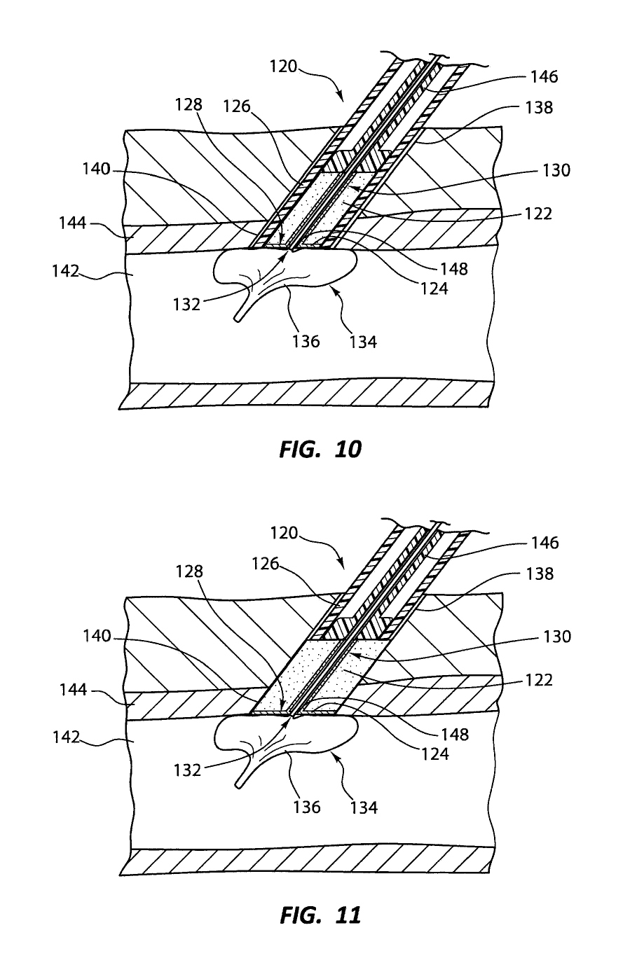

The bioresorbable carrier 14 may also include a valve 44 positioned within the tubular portion 30. The valve 44 may be configured to facilitate the passage of elongate devices, such as intravascular catheters, guide wires, locating devices (see FIG. 10), and the like, and may provide a hemostatic seal when no elongate device is positioned through the valve 44. The valve 44 may be integral to the bioresorbable carrier 14, which may be a monolithic structure molded as a single unit. For example, the bioresorbable carrier 14 may be molded from a bioresorbable polymer and flaps 46 may be formed by cutting slits in a relatively thin material layer molded within the tubular portion 30 of the bioresorbable carrier 14. In further embodiments, the valve may be formed separately from the tubular portion 30 of the bioresorbable carrier 14 and may be coupled to the tubular portion, such as by one or more of a mechanical interference fit and an adhesive. Likewise, in some embodiments, the tubular portion 30 may be formed separately from the base portion 32 and the tubular portion 30 and the base portion 32 may be coupled together, such as by one or more of a mechanical interference fit (e.g., a snap-fit) and an adhesive. Accordingly, the tubular portion 30 may be fixed relative to the base portion 32. Additionally, the valve 44 may be coupled to the tubular portion 30 of the bioresorbable carrier.

The valve 44 may include a plurality of flaps 46 that extend from the inner surface 38 of the tubular portion 30 of the bioresorbable carrier 14. The flaps 46 of the valve 44 may bend when a force is applied by an elongate device to allow passage therethrough. When an elongate device is not positioned through tubular portion 30 and the valve 44, the flaps 46 of the valve 44 may be biased to a closed position and provide a hemostatic seal, which may prevent blood flow through the tubular portion 30 of the bioresorbable carrier 14.

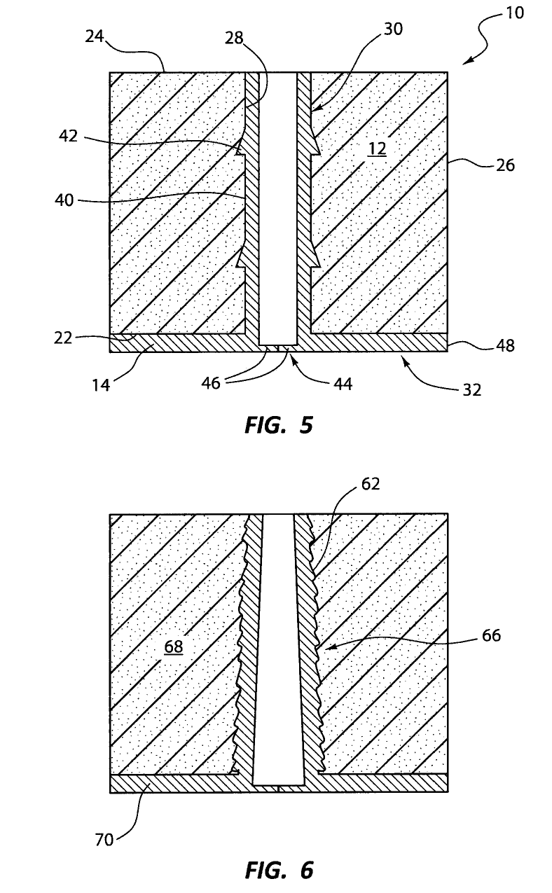

As shown in FIGS. 1 and 5, the collagen plug 12 may be positioned on the bioresorbable carrier 14 with the aperture 28 of the collagen plug 12 positioned over the outer surface 40 of the tubular portion 30 of the bioresorbable carrier 14. The first end surface 22 of the collagen plug 12 may be arranged in contact with the base portion 32 of the bioresorbable carrier 14. Additionally, the surface features 42 on the outer surface 40 of the tubular portion 30 of the bioresorbable carrier 14 may couple with the inner surface of the aperture 28 of the collagen plug 12 to maintain the position of the collagen plug 12 on the bioresorbable carrier 14. For example, the surface features 42 may include a plurality of angled or pointed rings extending from the outer surface 40 of the tubular portion 30 of the bioresorbable carrier 14 and into the surface within the aperture 28 of the collagen plug 12 to maintain the position of the collagen plug 12 on the bioresorbable carrier 14, as shown in FIG. 5. For another example, surface features 60 may include a texture on an outer surface of a tubular portion 66 that may grip a collagen plug 68 to maintain the position of the collagen plug 68 on a bioresorbable carrier 70, as shown in FIG. 6. In yet further embodiments, a bioresorbable adhesive or other bonding agent may be used to bond a collagen plug to a bioresorbable carrier.

The side surface 26 of the collagen plug 12 may be sized to correspond to the outer circumference 48 of the base portion 32 of the bioresorbable carrier 14, as shown in FIGS. 1 and 5. In some embodiments, however, the side surface 26 of the collagen plug 12 may extend beyond the outer circumference 48 of the base portion 32 of the bioresorbable carrier 14. Additionally, in some embodiments, the outer circumference 48 of the base portion 32 of the bioresorbable carrier 14 may extend beyond the side surface 26 of the collagen plug 12.

The size of the base portion 32 of the bioresorbable carrier 14 may be selected to completely cover or fill a vessel puncture. Meanwhile, the size of the collagen plug 12 may be selected to fill a cavity within a tissue tract. As the bioresorbable material of the bioresorbable carrier 14 (e.g., a bioresorbable polymer) may be substantially stiffer and more rigid than the collagen plug 12, the base portion 32 of the bioresorbable carrier 14 may prevent the collagen plug 12 from expanding into a vessel lumen, as the vessel puncture may be covered by the base portion 32 of the bioresorbable carrier 14. Yet, the relatively soft and resilient collagen plug 12 may effectively expand radially and fill the tissue tract.

While in some embodiments the hemostasis device 10 may comprise a bioresorbable carrier 14 with a substantially flat base portion 32 and a cylindrical tubular portion 30, as shown in FIGS. 1 and 3-6, additional shapes and configurations may also be utilized for a bioresorbable carrier.

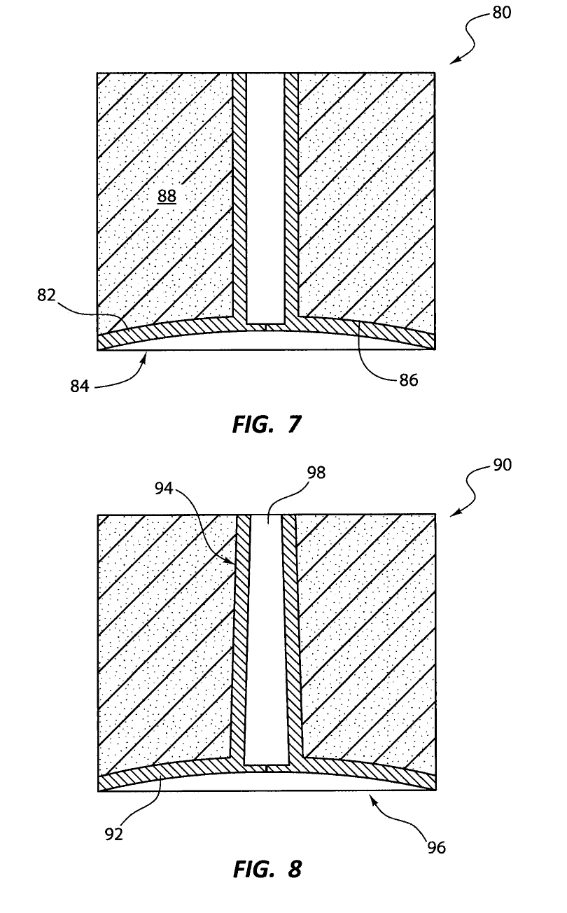

In some embodiments, as shown in FIG. 7, a hemostasis device 80 may comprise a bioresorbable carrier 82 having a dish-shaped (e.g., concave or convex) base portion 84. For example, the base portion 84 of the bioresorbable carrier 82 may have the shape and appearance of a suction cup. An end surface 86 of a collagen plug 88 may be shaped to correspond to the shape of the base portion 84 of the bioresorbable carrier 82. In further embodiments, however, a collagen plug may include a substantially planar end surface that may be deformed to conform to the shape of the base portion 84 of the bioresorbable carrier 82.

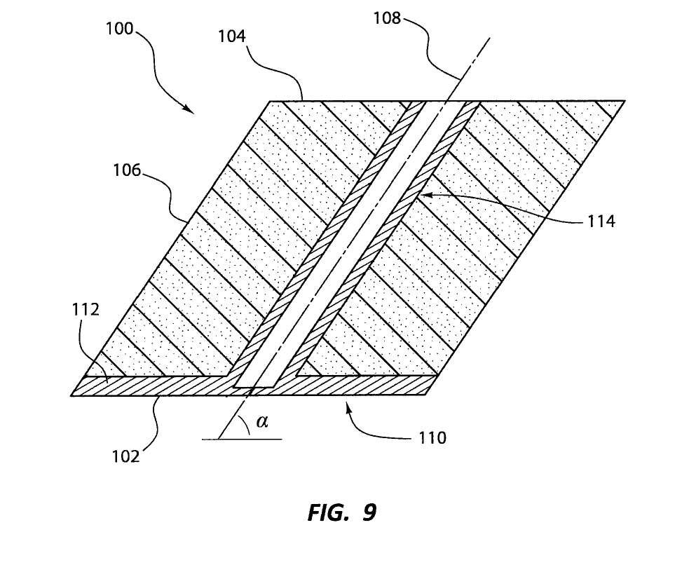

Additionally, a hemostasis device 90 may comprise a bioresorbable carrier 92 having a tapered tubular portion 94 with a tapered central aperture 98, as shown in FIG. 8. For example, the tubular portion 94 may have a generally frusto-conical shape (e.g., having a tapered shape, similar to a truncated cone). The tubular portion 94 may be tapered away from a base portion 96, the tubular portion 94 having a larger cross-sectional shape near the base portion 96 relative to regions of the tubular portion 94 that are distal from the base portion 96.

While in some embodiments the hemostasis device 10, 80, 90 may be shaped generally as a right circular cylinder, the curved outer surface arranged substantially perpendicular to the end surfaces as shown in FIGS. 1 and 5-8, additional shapes and configurations may be utilized for a hemostasis device in additional embodiments. For example, a hemostasis device may comprise a cross-section shaped generally as one or more of a circle, an ovoid, and an ellipse. Additionally, the end surfaces may be positioned at an acute angle relative to the outer surface of a hemostasis device.

In some embodiments, as shown in FIG. 9, a hemostasis device 100 may be generally cylindrical, but include end surfaces 102 and 104 positioned at an angle relative to the curved outer surface 106 and a central axis 108 of the hemostasis device 100. A first end surface 102 of the hemostasis device 100 may be oriented substantially parallel to an opposing second end surface 104 of the hemostasis device 100. Each of the first and second end surfaces 102 and 104 may be oriented at a non-perpendicular angle relative to the curved outer surface 106 and the central axis 108 of the hemostasis device 100. An angle a between each of the first and second end surfaces 102 and 104 and the central axis 108 of the hemostasis device 100 may be an acute angle. For example, the angle a may be an angle between about 45 degrees and about 90 degrees. For another example, the angle a may be an angle between about 60 degrees and about 85 degrees. Accordingly, a base portion 110 of a bioresorbable carrier 112 may be oriented at a non-perpendicular angle relative to a longitudinal axis of a tubular portion 114 of the bioresorbable carrier 112. The angle of the base portion 110 of the bioresorbable carrier 112 may be oriented to substantially match the angle of a vascular opening relative to an orientation of a correlating vascular wall. That is to say, the hemostasis device 100 may be shaped and configured to correspond to the shape of an opening, such as a vascular opening, to be closed by the hemostasis device 100.

In many operations, a tissue tract providing access to a vascular opening may be oriented at a non-perpendicular angle to the vascular wall wherein a catheter or other device is inserted. Such an angled tissue tract may facilitate the insertion of a catheter into a vascular lumen, as the catheter may not be required to bend at a right angle. Accordingly, a hemostasis device that is shaped to correspond to the angle of a tissue tract relative to an underlying vascular wall may provide an effective and complete closure of a vascular opening.

Referring to FIG. 10, for delivery of a hemostasis device 120 into a patient, a collagen plug 122 and a bioresorbable carrier 124 of the hemostasis device 120 may be positioned at a distal end of a delivery tube 126. The delivery tube 126 may compress and elastically deform the collagen plug 122 and the bioresorbable carrier 124. For example, the outer circumference of the collagen plug 122 may be compressed toward a central axis of the hemostasis device 120. Additionally, a base portion 128 of the bioresorbable carrier 124 may be elastically deformed within the delivery tube 126.

As used herein, the terms “elastic deformation,” “elastically deform,” and “elastically deformed” indicate that a deformation (i.e., the change in shape) of a structure is elastic (i.e., reversible) and the structure may resiliently return to its original shape, or at least close to its original shape, once external forces are no longer applied.

Prior to full insertion of the hemostasis device 120 into a patient, a locating device 134 may be positioned through a tubular portion 130 and a valve 132 of the bioresorbable carrier 124. For example, the locating device 134 may comprise an inflatable balloon 136 at a distal end that may be positioned through the tubular portion 130 of the bioresorbable carrier and the valve 132.

For the closure of a vessel puncture 140 (e.g., an artery puncture or a vein puncture) of a vessel 144, the distal end of the locating device 134 may be advanced through a tissue tract 138 and a vessel puncture 140 and into a vessel lumen 142. The balloon 136 may then be inflated by delivering a volume of inflation fluid from an inflation fluid source (not shown). The locating device 134 may then be retracted (e.g., withdrawn proximally) to bring the inflated balloon 136 into contact with an inner surface of the vessel 144 adjacent to the vessel puncture 140. Accordingly, the inflated balloon 136 may provide a temporary seal with the vessel to limit blood flow through the vessel puncture from within the vessel lumen.

The hemostasis device 120 may then be advanced to an outer surface of the vessel 144. The delivery tube 126 may then be retracted while the base portion 128 of the bioresorbable carrier 124 is held in position against the outer surface of the vessel 144 with a plunger 146 positioned within the delivery tube 126. As the delivery tube 126 is withdrawn from over the collagen plug 122 and the bioresorbable carrier 124, the collagen plug 122 and the base portion 128 of the bioresorbable carrier 124 may elastically deform, as shown in FIG. 11. For example, a cross-sectional size of the collagen plug 122 and the base portion 128 of the bioresorbable carrier 124 may resiliently expand (i.e., the collagen plug 122 and the base portion 128 of the bioresorbable carrier 124 may expand radially) within the tissue tract 138.

After the delivery tube 126 has been withdrawn from over the collagen plug 122 and the bioresorbable carrier 124, the collagen plug 122 and bioresorbable carrier 124 may be positioned to provide a hemostatic seal. Next, the balloon 136 of the locating device 134 may be deflated by withdrawing the volume of inflation fluid back into the inflation fluid source, and the locating device 134 may be removed. Optionally, the collagen plug 122 and the bioresorbable carrier 124 may be attached to surrounding tissue with sutures (not shown).

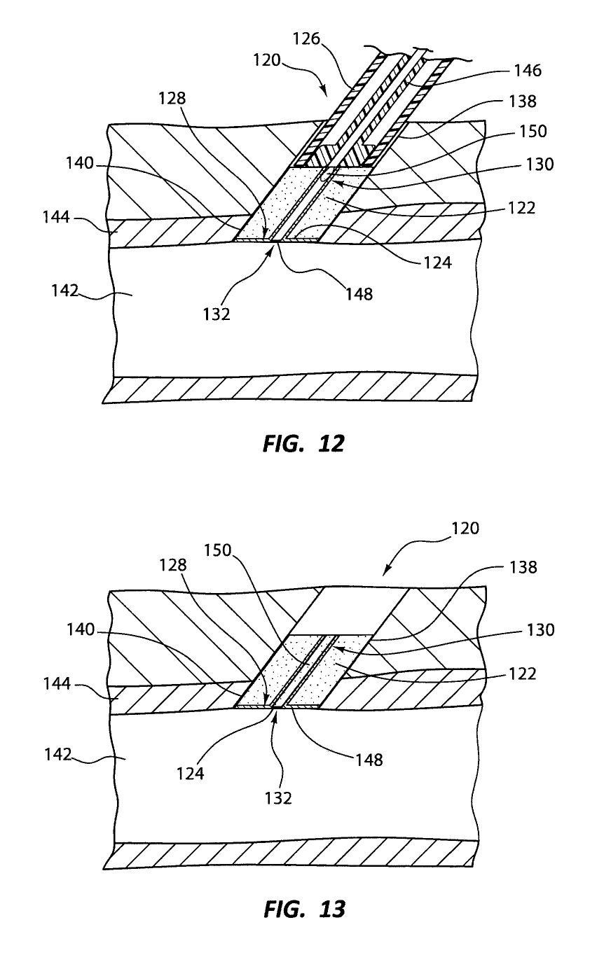

As shown in FIG. 12, the locating device 134 may be retracted from the tubular portion 130 of the bioresorbable carrier 124, which may assist in collapsing the deflated balloon 136. Accordingly, the size and shape of the tubular portion 130 of the bioresorbable carrier 124 may be selected to assist in collapsing the corresponding locating device 134. For example, the tubular portion 130 may have a tapered shape that assists in collapsing a locating device 134.

After the locating device 134 is removed from the valve 132 located in the tubular portion 130 of the bioresorbable carrier 124, flaps 148 of the valve 132 may be biased to come together and close the tubular portion 130 of the bioresorbable carrier 124. In some embodiments, the valve 132 may provide a hemostatic seal that will prevent blood flow through the tubular portion 130 of the bioresorbable carrier 124. In further embodiments, the valve 132 may restrict blood flow through the tubular portion 130 of the bioresorbable carrier 124 without providing a complete hemostatic seal.

Referring to FIG. 13, after the locating device 134 has been removed from the bioresorbable carrier 124, a bioresorbable plug 150 may be inserted into the tubular portion 130 of the bioresorbable carrier 124. For example, the bioresorbable plug 150 may be attached to the distal end of the locating device 134, and as the locating device 134 is retracted through the tubular portion 130 of the bioresorbable carrier 124 the bioresorbable plug 150 may be lodged into the tubular portion 130 of the bioresorbable carrier 124. For another example, after the locating device 134 has been removed from the bioresorbable carrier 124 a bioadhesive may be injected into the tubular portion 130 of the bioresorbable carrier 124, which may cure to form the bioresorbable plug 150.

After the tubular portion 130 of the bioresorbable carrier 124 has been sealed, either by the valve 132, the bioresorbable plug 150, or a combination thereof. The sealing procedure may be complete, and a hemostatic seal of the vessel puncture 140 may be provided. When installed against the vessel 144, the base portion 128 of the bioresorbable carrier 124 may assist in mechanically sealing the vessel, such as by the application of pressure applied by the physician in a distal direction during installation of the hemostasis device. Additionally, the base portion 128 of the bioresorbable carrier 124 may prevent collagen from entering the interior of the vessel 144 and may prevent the collagen plug 122 from being exposed to blood flow.

Referring to FIGS. 1-5, a method of preparing the hemostasis device 10 may comprise providing the bioresorbable carrier 14, providing the collagen plug 12 and positioning the collagen plug 12 on the bioresorbable carrier.

In one embodiment, to provide the bioresorbable carrier 14, a molding process may be utilized. A mold (not shown) may comprise a cavity that corresponds to the tubular portion 30, the base portion 32, and the valve 44. A bioresorbable polymer may be injected into the mold, where the bioresorbable polymer may cure to form a bioresorbable carrier precursor. The bioresorbable carrier precursor may include the fully formed tubular portion 30 and base portion 32. The central passage of the tubular portion 30 may be closed off by a material layer, which may be utilized to form the valve 44. A plurality of slits may be cut into the material layer of the bioresorbable carrier precursor to form the flaps 46 of the valve 44 and to complete the bioresorbable carrier 14.

To provide the collagen plug 12, a sheet of collagen material may be provided. The collagen material may comprise a sponge material and may comprise any number of different sealing materials alone or in combination. A punch set may then be pressed into the sheet of collagen material and form concentric circular cuts in the sheet of collagen material. An annular portion of the sheet of collagen material located between the concentric circular cuts may then be removed from the sheet of collagen material to provide the collagen plug 12. Optionally, human coagulation factors, such as fibrinogen and thrombin may be applied to the collagen plug 12.

The materials used for collagen plug 12 may include any desired bioresorbable material, such as those disclosed in U.S. patent application Ser. No. 13/391,878, filed on 23 Feb. 2012, and titled “Single Piece, Dual Component Sealing Pad and Methods,” which application is incorporated herein in its entirety by this reference.

The preceding description has been presented only to illustrate and describe example embodiments of the invention. It is not intended to be exhaustive or to limit the invention to any precise form disclosed. Many modifications and variations are possible in light of the above teachings. It is intended that the scope of the invention be defined by the following claims.

What is claimed is:

1. A hemostasis device, the device comprising:
a collagen plug comprising a body having a first end surface and an aperture extending through the body and the first end surface of the collagen plug;
a bioresorbable carrier comprising a tubular portion extending through the aperture of the body of the collagen plug, and a base portion extending radially from an end of the tubular portion, the base portion positioned adjacent to the first end surface of the collagen plug.

2. The device of claim 1, wherein the body of the collagen plug further comprises a second end surface, the aperture extending through the body extending from the first end surface to the second end surface and the tubular portion of the bioresorbable carrier extending through the aperture from the first end surface to the second end surface.

3. The device of claim 2, wherein the tubular portion of the bioresorbable carrier comprises a tapered central aperture.

4. The device of claim 1, wherein the body of the collagen plug is substantially cylindrical.

5. The device of claim 4, wherein the base portion of the bioresorbable carrier has a substantially circular outer circumference.

6. The device of claim 1, wherein the base portion of the bioresorbable carrier is substantially flat, having substantially planar major surfaces.

7. The device of claim 1, wherein the base portion of the bioresorbable carrier is concave, having dished major surfaces.

8. The device of claim 1, wherein the base portion of the bioresorbable carrier is angled relative to the tubular portion, and major surfaces of the base portion are oriented at a non-perpendicular angle relative to a longitudinal axis of the tubular portion.

9. The device of claim 1, wherein the bioresorbable carrier comprises an integral valve within the tubular portion of the bioresorbable carrier for providing a hemostatic seal.

10. The device of claim 9, wherein the integral valve is positioned at an end of the tubular portion proximate to the base portion of the bioresorbable carrier.

11. The device of claim 9, wherein the integral valve comprises a plurality of flaps.

12. The device of claim 1, wherein the bioresorbable carrier is comprised of a bioresorbable polymer.

13. The device of claim 1, wherein an outer diameter of the tubular portion of the bioresorbable carrier comprises surface features configured to maintain the position of the collagen plug relative to the bioresorbable carrier.

14. The device of claim 1, further comprising a delivery tube, the bioresorbable carrier and collagen plug located in a distal end of the delivery tube.

15. The device of claim 14, wherein the base portion of the bioresorbable carrier is elastically deformed within the distal end of the delivery tube.

16. A method of manufacturing a hemostasis device, the method comprising:
providing a collagen plug comprising a body having a first end surface and an aperture extending through the body and the first end surface of the collagen plug;
providing a bioresorbable carrier comprising a tubular portion and a base portion extending radially from an end of the tubular portion;
positioning the tubular portion of the bioresorbable carrier within the aperture of the collagen plug and positioning the base portion of the bioresorbable carrier adjacent to the first end surface of the collagen plug.

17. The method of claim 16, further comprising forming an integral hemostatic seal within the tubular portion of the bioresorbable carrier.

18. The method of claim 16, wherein providing a bioresorbable carrier comprising a tubular portion comprises providing a bioresorbable carrier comprising a tubular portion with a tapered central aperture.

19. The method of claim 16, wherein providing a bioresorbable carrier comprising a tubular portion and a base portion extending radially from an end of the tubular portion comprises providing a bioresorbable carrier comprising a tubular portion and a concave base portion having dished major surfaces extending radially from an end of the tubular portion.

20. A component for a hemostasis device, the component comprising:
a bioresorbable carrier comprising:
a tubular portion comprising an inner surface, an outer surface, a first end and a second end;
a base portion extending radially from the second end of the tubular portion;
a valve located within the tubular portion configured to facilitate the passage of an elongate device through the tubular portion and to provide a hemostatic seal of the tubular portion when an elongate device is not positioned through the valve;
wherein the base portion and the outer surface of the tubular portion are configured to receive a collagen plug thereon.
